(19) United States
(12) Patent Application Publication
(10) Pub. No.: US 2009/0104675 A1
(43) Pub. Date: Apr. 23, 2009

(54) METHOD FOR PRODUCING LACTIC ACID

(75) Inventors: Ikuo Yamaguchi, Okazaki-shi (JP); Osamu Saotome, Nissin-shi (JP); Shigeru Kuromiya, Nagoya-shi (JP); Tohru Ohnishi, Toyota-shi (JP); Noriko Yasutani, Nagoya-shi (JP); Satoshi Saitoh, Nishikamo-gun (JP); Makoto Mouri, Seto-shi (JP); Mitsuru Nakano, Nagoya-shi (JP); Arimitsu Usuki, Nagoya-shi (JP); Nobuhiro Ishida, Aichi-gun (JP); Kenro Tokuhiro, Aichi-gun (JP); Eiji Nagamori, Nissin-shi (JP); Haruo Takahashi, Ohgaki-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: TOYOTA JIDOSHA KABUSHIKI KAISHA

(21) Appl. No.: 12/275,099

(22) Filed: Nov. 20, 2008

Related U.S. Application Data
(62) Division of application No. 10/592,384, filed on Sep. 12, 2006.

(30) Foreign Application Priority Data
Sep. 13, 2004 (JP) 2004-265655

(51) Int. Cl. C12P 7/56

(52) U.S. Cl. 435/139

(57) ABSTRACT

Lactic acid with high optical purity that has not previously been achieved is produced.

It has been found that the optical purity of lactic acid is reduced as the racemization reaction of lactic acid proceeds when lactic acid coexists with glycerol. By reducing the amount of glycerol prior to concentrating lactic acid by heating, the optical purity of lactic acid after concentration by heating can be maintained at a high level.

### TECHNICAL FIELD

The present invention relates to a method for producing lactic acid whereby lactic acid, which is a material used for producing polylactic acid and the like, can be produced with high optical purity.

### BACKGROUND ART

Polylactic acid is a polymer that is degradable in vivo and excellent in terms of mechanical properties and the like, and thus it has been used in the field of medicine. In addition, polylactic acid has been expected to be utilized for a variety of applications from the viewpoint of environmental protection, since it is also degradable in the natural environment.

Examples of a method for producing polylactic acid include a method of direct dehydration condensation of lactic acid as a starting material, a method of dealcoholization condensation of lactate ester, and a method of ring-opening polymerization of lactide. With any of these methods, polylactic acid excellent in terms of physical properties can be produced using lactic acid with high optical purity.

An example of a method for producing lactic acid is a fermentation method using a microorganism that has a system for lactic acid biosynthesis or a microorganism to which a system for lactic acid biosynthesis is imparted. It is considered that, using such fermentation method, polylactic acid production using lactic acid with high optical purity as a starting material can be achieved as described above using a strain that produces only either L-lactic acid or D-lactic acid due to the gene structure thereof.

However, when polylactic acid is required to have more excellent physical properties, it has been difficult to prepare lactic acid with sufficient optical purity via a conventional fermentation method, even using a strain that produces only either L-lactic acid or D-lactic acid.

### DISCLOSURE OF THE INVENTION

Thus, in view of the actual situation described above, it is an object of the present invention to provide a method for producing lactic acid whereby it is possible to produce lactic acid with high optical purity, which is, for example, also available as a starting material for polylactic acid excellent in terms of physical properties.

As a result of intensive studies to achieve the above object, inventors of the present invention have found that the optical purity of lactic acid is reduced as the racemization reaction of lactic acid proceeds when lactic acid coexists with glycerol. This has led to the completion of the present invention.

That is, the present invention includes the following:

(1) a method for producing lactic acid, comprising a step of concentrating lactic acid in a solution containing a reduced amount of glycerol by heating;

(2) the method for producing lactic acid described in (1), further comprising a step of preparing the solution by lactic acid fermentation using a microorganism having a reduced capacity for glycerol production;

(3) the method for producing lactic acid described in (2), wherein the microorganism is a variant, in which expression of at least one gene involved in glycerol production is suppressed;

(4) the method for producing lactic acid described in (3), wherein the variant is a lactic acid-producing microorganism, in which a gene encoding glycerol-3-phosphate dehydrogenase is disrupted;

(5) the method for producing lactic acid described in (4), wherein the lactic acid-producing microorganism is a microorganism classified as a member of the genus Saccharomyces.

(6) the method for producing lactic acid described in (1), wherein the amount of glycerol relative to that of lactic acid in the solution is 3.5% by weight or less, and more preferably 0.1% by weight or less.

(7) the method for producing lactic acid described in (1), further comprising a step of preparing the solution by lactic acid fermentation using a microorganism and a step of removing glycerol from the solution;

(8) the method for producing lactic acid described in (7), wherein the amount of glycerol relative to that of lactic acid in the solution is 3.5% by weight or less, and more preferably, 0.1% by weight or less during the step of removing glycerol;

(9) a variant, which is obtained by mutagenizing a lactic acid-producing microorganism such that the amount of glycerol produced is reduced;

(10) the variant described in (9), wherein the lactic acid-producing microorganism is a microorganism classified as a member of the genus Saccharomyces;

(11) the variant described in (9), wherein the amount of glycerol produced is reduced by disrupting a gene encoding glycerol-3-phosphate dehydrogenase; and

(12) the variant described in (9), which is obtained by introducing variation into a lactic acid-producing microorganism such that the amount of glycerol produced relative to that of lactic acid is reduced to 3.5% by weight or less, and more preferably to 0.1% by weight or less.

Further, the inventors of the present invention have found that production efficiency of lactic acid is improved using a microorganism having a reduced capacity for glycerol production in lactic acid fermentation. This has led to the completion of the present invention. That is, the present invention includes the following:

(13) a method for producing lactic acid, comprising a step of producing lactic acid by lactic acid fermentation using a microorganism having a reduced capacity for glycerol production;

(14) the method for producing lactic acid described in (13), wherein the organism is a variant, in which expression of at least one gene involved in glycerol production is suppressed;

(15) the method for producing lactic acid described in (14), wherein the variant is a lactic acid-producing microorganism, in which glycerol-3-phosphate dehydrogenase is disrupted;

(16) the method for producing lactic acid described in (15), wherein the lactic acid-producing microorganism is a microorganism classified as a member of the genus Saccharomyces; and

(17) the method for producing lactic acid described in (15), wherein variation is introduced into the lactic acid-producing microorganism such that the amount of glycerol produced relative to that of lactic acid is reduced by 3.5% by weight or more, and more preferably by 0.1% by weight or more.

This description includes part or all of the contents as disclosed in the description of Japanese Patent Application No. 2004-265655, which is a priority document of the present application.

### BRIEF DESCRIPTION OF THE DRAWINGS

FIG. 1 shows a chromatogram obtained as a result of GC-MS analysis using a solution containing L-lactic acid and glycerol.

FIG. 2 shows MS spectra obtained as a result GC-MS analysis using a solution containing L-lactic acid and glycerol.

FIG. 3 shows chromatograms obtained as a result of GC-MS analysis using a solution containing L-lactic acid and ethylene glycol.

FIG. 4 shows MS spectra obtained as a result GC-MS analysis using a solution containing L-lactic acid and ethylene glycol.

### BEST MODE FOR CARRYING OUT THE INVENTION

The present invention will hereafter be described in greater detail with reference to the drawings.

The method for producing lactic acid according to the present invention includes a step of concentrating lactic acid in a solution containing a reduced amount of glycerol by heating. Particularly, the present invention is applied to lactic acid production via a fermentation method. A fermentation method is a phenomenon in which saccharide in a medium generates lactic acid due to the action of microorganisms. In the following descriptions, microorganisms having the capacity for lactic acid generation and microorganisms to which such capacity is imparted are collectively referred to as “lactic acid-producing bacteria”

Also, in the present invention, “reducing the amount of glycerol” means reducing the capacity for glycerol production of a lactic acid-producing microorganism by a fermentation method, removing and/or degrading glycerol that has been produced by a lactic acid-producing microorganism, or both thereof. It is revealed that racemization of lactic acid proceeds based on the following reaction when lactic acid and glycerol coexist.

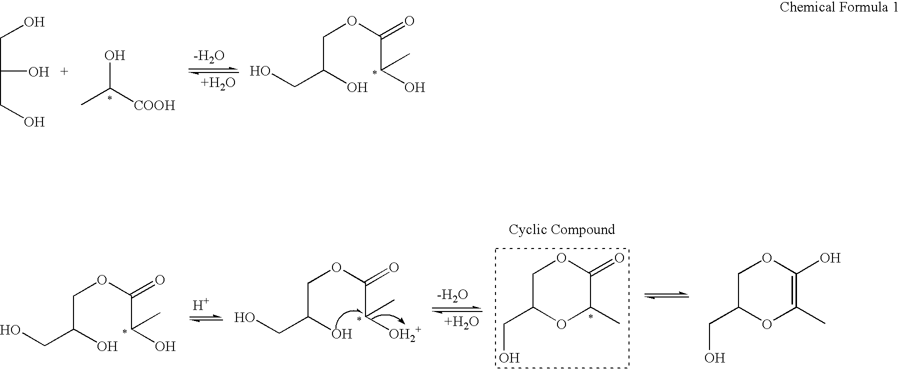

In addition, the aforementioned reaction proceeds due to thermal energy that is added in a step of concentrating lactic acid that has been generated by heating, esterification by heating, or heating distillation, resulting in disadvantageously reduced optical purity. Thus, by reducing the amount of glycerol prior to a step of heating lactic acid that has been generated, lactic acid with high optical purity can be obtained.

As a method for reducing the amount of glycerol, a method for reducing the capacity for glycerol production of a lactic acid-producing microorganism by a fermentation method (method 1) and a method for removing and/or degrading glycerol that has been produced by a lactic acid-producing microorganism (method 2) will hereafter be described in that order.

### Method 1

The following methods 1) to 8) can be used when reducing the capacity for glycerol production of a lactic acid-producing microorganism:

1) disrupting a gene involved in glycerol production, which a lactic acid-producing microorganism has;
2) suppressing expression of a gene involved in glycerol production;
3) inhibiting activity of a protein encoded by a gene involved in glycerol production;
4) improving the capacity for glycerol metabolism and degradation;
5) suppressing glycerol secretion outside the cell membrane;
6) promoting glycerol uptake inside the cell membrane;
7) obtaining a mutant strain in which the amount of glycerol produced is reduced; and
8) adding a compound which results in reduction in the amount of glycerol produced to a culture medium. The capacity for glycerol production of a lactic acid-producing microorganism may be reduced by any one of or by a combination of two or more of methods 1) to 8) described above.

Here, examples of a lactic acid-producing microorganism that can be used for the method for producing lactic acid according to the present invention include bacteria, yeasts, and fungi that are microorganisms having the capacity for lactic acid generation. Examples of such bacteria include Lactobacillus bacteria, Streptococcus bacteria, Bacillus bacteria, Leuconostoc bacteria, and Pediococcus bacteria. Examples of such yeasts include Kluyveromyces yeasts. Examples of such fungi include Rhizopus fungi and Aspergillus fungi. Particularly preferably, these lactic acid-producing microorganisms used are microorganisms having the capacity for homolactic fermentation.

In addition, a microorganism to which the capacity for lactic acid generation is imparted means a microorganism that does not originally have the capacity for lactic acid generation but rather was modified to have such capacity by a genetic engineering technique. Examples thereof include a yeast mutant obtained by introducing a gene involved in lactic acid generation into Saccharomyces cerevisiae. Further, in addition to such yeast mutants, bacteria, yeasts, and fungi that do not have the capacity for lactic acid generation can be used after introducing a gene involved in lactic acid generation thereinto. Specific examples of such microorganisms can be classified as members of the genera Saccharomyces, Schizosaccharomyces, Kluyveromyces, Pichia, Hansenula, Candida, Trichosporon, or Yamadazyma. Examples of such bacteria include Escherichia coli bacteria, Zymomonas bacteria, and coryneform group bacteria. Examples of such fungi include Rhizopus bacteria,Aspergillus bacteria, and Mucor bacteria.

Examples of a gene involved in lactic acid generation include a gene (LDH gene) encoding a protein that has lactate dehydrogenase activity. A variety of homologues of lactate dehydrogenase (LDH) are found depending on the species of organism or in vivo. LDHs used in the present invention include not only naturally derived LDHs but also chemically synthesized or genetically engineered, artificially synthesized LDHs. Preferably, such LDHs are derived from eukaryotes such as fungi or prokaryotes such as Lactobacillus helveticus, Lactobacillus casei, Kluyveromyces thermotolerans, Torulaspora delbrueckii, Schizosaccharomyces pombe, and Rhizopus oryzae. More preferably, they are derived from higher eukaryotes such as plants, animals, and insects. An example thereof is a bovine LDH (L-LDH). Genes of the above organisms are introduced into microorganisms such as the aforementioned yeasts that do not originally have the capacity for lactic acid generation, such that the capacity for lactic acid generation can be imparted to such microorganisms. In the method for producing lactic acid according to the present invention, the thus obtained microorganisms to which the capacity for lactic acid generation has been imparted can widely be used.

### 1) Method for Disrupting a Gene Involved in Glycerol Production Contained in a Lactic Aid-Producing Microorganism

Glycerol production involves acetaldehyde generated in a glycolytic pathway in a microorganism being removed from the alcohol dehydrogenase reaction system, such that fermentation conversion that results in NADH oxidation using glycerol-3-phosphate dehydrogenase is induced, leading to generation and accumulation of glycerol. A gene involved in glycerol production is a gene encoding an enzyme that contributes to one of the reactions for glycerol production described above.

Examples of a gene involved in glycerol production include a glycerol-3-phosphate dehydrogenase gene, a glycerol-1-phosphate dehydrogenase gene, and a glycerokinase gene. More specifically, such examples include GPD1 and GPD2 genes (glycerol-3-phosphate dehydrogenase genes), RHR2 and HOR2 genes (glycerol-1-phosphate dehydrogenase genes), and GPP1 and GPP2 genes (glycerokinase genes) for Saccharomyces cerevisiae.

In addition, in the case of GPD1 and GPD2 genes for Saccharomyces cerevisiae, glycerol production can be reduced in a strain in which either one of or both of the genes have been disrupted (Nissen T. L. et al., Yeast 16, 463-474 (2000)). In the case of RHR2 and HOR2 genes for Saccharomyces cerevisiae, glycerol production can be reduced in a strain in which both of the genes have been disrupted (Pahlman A. K. et al, J. Biol. Chem. 276, 3555-3563 (2001)).

Examples of a method for disrupting the aforementioned genes in a lactic acid-producing microorganism include, but are not particularly limited to, a method for deleting such genes from the genome and a method for inserting foreign DNA fragments into such genes.

As described above, glycerol production in a lactic acid-producing microorganism can be suppressed by disrupting genes involved in glycerol production.

### 2) Method for Suppressing Expression of a Gene Involved in Glycerol Production

Methods for suppressing expression of a gene involved in glycerol production exclude methods for disrupting the aforementioned genes, and include methods for suppressing the expression of the genes. Examples of a method for suppressing expression of a gene include a method for suppressing transcription of the aforementioned genes, a method for inhibiting translation of the genes after the transcription thereof, and a method for selectively degrading the mRNA of the genes.

More specifically, examples of a method for suppressing transcription of the aforementioned genes include a method for deleting transcriptional control regions of the genes from the genome and a method for inserting foreign DNA fragments into transcriptional control regions of the genes. In addition, by introducing nucleic acid encoding an RNA decoy into a cell, expression of the aforementioned genes can be suppressed at the transcription level. Such RNA decoy is a gene encoding a binding protein of a transcriptional factor or RNA comprising a sequence of a binding site of a transcriptional factor or a sequence analogous thereto. These are introduced into a cell as a “decoy,” so that the function of a transcriptional factor is suppressed.

Meanwhile, examples of a method for inhibiting translation of the aforementioned genes include an antisense RNA method. The antisense RNA method indicates a method for introducing antisense RNA that is hybridized to a part or all of mRNA, or a method for introducing a DNA fragment that encodes such antisense RNA into a host genome. Antisense RNA is RNA that has a nucleotide sequence complementary to that of the mRNA of interest, so that these RNAs constitute a double strand, resulting in suppression of expression of a gene encoded by the mRNA at the translation level. In addition, instead of such antisense RNA, antisense DNA can be used so that expression of a novel gene can be suppressed at the transcription level. In any case, an antisense sequence that can be used comprises any nucleic acid substance that blocks gene translation or transcription. Examples thereof include DNA, RNA, or arbitrary pseudo-nucleic acid substances. Thus, an antisense nucleic acid (oligonucleotide) sequence may be designed in a manner such that the sequence is complementary to a part of the sequence of a novel gene, the expression of which is suppressed. Also, a molecular analogue of an antisense oligonucleotide can be used. Such molecular analogue has high stability, distribution specificity, and the like. Examples of such molecular analogue include chemically reactive groups obtained by allowing, for example, iron-binding ethylenediaminetetraacetic acid bind to an antisense oligonucleotide.

Further, expression of the aforementioned genes can be suppressed using ribozymes at the translation level. Here, ribozymes include those that cleave mRNA of a specific protein and inhibit translation of such protein. Ribozymes can be designed based on the arrangement of a gene encoding a specific protein. For instance, hammerhead ribozymes can be designed by a method described in FEBS letter, 228; 228-230 (1988). Also, in addition to hammerhead ribozymes, ribozymes such as hairpin and delta ribozymes can be used in the present invention, as long as they cleave mRNA of a specific protein and inhibit translation of such protein.

Examples of a method for selectively degrading mRNA of the aforementioned genes include a method utilizing RNA interference. RNA interference is a phenomenon in which intracellular RNA that forms a double strand (hereafter to be referred to as “double-stranded RNA” or “dsRNA”) causes degradation of endogenous mRNA that has a sequence homologous to that of the RNA, resulting in specifically suppressed gene expression based on such mRNA. RNA interference can be referred to as RNAi. A gene in which the principle of RNA interference is used is designed based on a nucleotide sequence of a gene of interest, the expression of which is suppressed, in a manner such that double-stranded RNA such as hairpin dsRNA is formed in a host.

As described above, by suppressing expression of a gene involved in glycerol production, glycerol production in a lactic acid-producing microorganism can be suppressed.

### 3) Method for Inhibiting Activity of a Protein Encoded by a Gene Involved in Glycerol Production

Glycerol production in a lactic acid-producing microorganism can be suppressed by inhibiting activities of enzymes encoded by the aforementioned genes involved in glycerol production. Specifically, antibodies against such enzymes or substances that specifically act on such enzymes can be used.

Such antibodies can be obtained by applying a known method and are not limited in terms of origin, class (monoclonal or polyclonal), or shape thereof, on the condition that they inhibit activities of the aforementioned enzymes. For instance, as long as such antibodies recognize the aforementioned enzymes as antigens and bind thereto, examples of the antibodies that can adequately be used include, but are not particularly limited to, murine antibodies, rat antibodies, rabbit antibodies, and sheep antibodies. The antibodies may be either polyclonal or monoclonal antibodies. However, monoclonal antibodies are preferable in terms of stable production of homogenous antibodies. Polyclonal or monoclonal antibodies can be produced by a method known by a person skilled in the art.

Hybridomas that can produce monoclonal antibodies can basically be produced using a known method as described below. That is, such hybridomas can be produced in a manner such that an antigen of interest and a cell that can express such antigen are used as sensitizing antigens for immunization in accordance with a conventional immunization procedure, and then the obtained immunocyte is fused with a known parent cell by a conventional cell fusion method, followed by screening of a monoclonal antibody-producing cell (hybridoma) based on a conventional screening method. Hybridomas can be produced according to, for example, a method of Milstein et al. (Kohler. G. and Milstein, C., Methods Enzymol. (1981) 73: 3-46)

Meanwhile, an inhibitor that can be used is a substance having a function of specifically inhibiting activities of enzymes encoded by the aforementioned genes involved in glycerol production.

As described above, glycerol production in a lactic acid-producing microorganism can be suppressed by inhibiting activities of enzymes encoded by genes involved in glycerol production.

### 4) Improvement of the Capacity for Glycerol Metabolism and Degradation

To improve the capacity for glycerol metabolism and degradation, a method for causing excessive expression of a gene involved in glycerol metabolism can be used. Examples of a gene involved in glycerol metabolism include a glycerol phosphoenzyme gene and a glycerol-3-phosphate dehydrogenase gene. In addition, examples thereof for Saccharomyces cerevisiae include a glycerol phosphoenzyme gene (GUT1) a glycerol-3-phosphate dehydrogenase gene (GUT2), a glycerol dehydrogenase gene (GCY1), and dihydroacetone phosphoenzyme gene (DAK1).

A method for introducing the aforementioned genes into lactic acid-producing bacteria is not particularly limited. DNA fragments, plasmids (DNA), viruses (DNA), retrotransposons (DNA), and artificial chromosomes (YAC) in a linear form or the like, into which the above genes are incorporated, are selected in accordance with forms of foreign gene introduction (extrachromosomal or intrachromosomal), such that recombinant vectors can be produced and introduced into lactic acid-producing bacteria.

As described above, glycerol production in a lactic acid-producing microorganism can be suppressed by improving the capacity for glycerol metabolism and degradation.

### 5) Suppression of Glycerol Secretion Outside a Cell Membrane

To suppress glycerol secretion outside a cell membrane, a method for disrupting a gene encoding a channel for glycerol secretion outside a cell membrane can be used. Examples of such gene include an FPS1 gene in Saccharomyces cerevisiae.

Examples of a method for disrupting the aforementioned genes in a lactic acid-producing microorganism include, but are not particularly limited to, a method for deleting the genes from the genome, a method for inserting foreign DNA fragments into the genes, and a method for introducing variation that results in reduced activities of expression proteins of the genes.

In addition, in accordance with the method described in the above “2) a method for suppressing expression of a gene involved in glycerol production,” a method for suppressing expression of a gene encoding a channel for glycerol secretion outside a cell membrane may be selected. Similarly, in accordance with the method described in the above “3) a method for inhibiting activity of a protein encoded by a gene involved in glycerol production,” a method for inhibiting activity of a protein encoded by a gene encoding a channel for glycerol secretion outside a cell membrane may be selected.

As described above, glycerol production in a lactic acid-producing microorganism can be suppressed by suppressing glycerol secretion outside the cell membrane.

### 6) Promotion of Glycerol Uptake Inside the Cell Membrane

To promote glycerol uptake inside the cell membrane, a method for causing excessive expression of a gene encoding a pump for glycerol uptake can be used. Examples of such gene include GUP1 and GUP2 genes in Saccharomyces cerevisiae.

As described above, glycerol production in a lactic acid-producing microorganism can be suppressed by promoting glycerol uptake inside the cell membrane.

7) Obtaining of a Mutant Strain in which the Amount of Glycerol Produced is Reduced

To obtain a mutant strain in which the amount of glycerol produced is reduced, any mutation method may be used as a method for obtaining yeast mutants. Examples thereof include physical methods such as ultraviolet radiation and radiation and a chemical method wherein yeast is suspended in a modifying agent such as a solution of ethylmethane sulfonate, N-methyl-N-nitroguanidine, nitrite, acridine dye, or the like. Also, a yeast mutant of interest can be obtained by spontaneous mutation, though it can be obtained at a lower frequency.

In the mutant strain obtained as described above in which the amount of glycerol produced is reduced, glycerol production is suppressed. Here, examples of such mutant strain may include a strain in which the amount of glycerol produced is reduced as a result of mutation of a gene involved in glycerol biosynthesis, glycerol metabolism, glycerol secretion outside the cell, or glycerol uptake inside the cell membrane. The strain is not limited in terms of the site into which mutation is introduced.

8) Addition of a Compound that Results in Reduction in the Amount of Glycerol Produced to a Culture Medium

Concerning the addition of a compound that results in reduction in the amount of glycerol produced to a culture medium, it has been known that the amount of glycerol produced is reduced by adding inositol, catechin, sodium disulfite, an antioxidant, or the like to a culture medium in the case of Saccharomyces cerevisiae (Caridi, A. (2002). Protective agents used to reverse the metabolic changes induced in wine yeasts by concomitant osmotic and thermal stress, Lett Appl Microbiol 35, 98-101). In addition, other compounds that cause reduction in the amount of glycerol produced may be added.

As described above, glycerol production in a lactic acid-producing microorganism can be suppressed by adding a compound that results in reduction in the amount of glycerol produced to a culture medium.

Also, in methods 1) to 8) described above, culture conditions and medium compositions for lactic acid-producing bacteria are not particularly limited, so that common culture conditions and medium compositions can be applied to such methods. For instance, when using Saccharomyces cerevisiae strain TC38 (a strain in which GPD1 and GPD2 genes are disrupted), to which the capacity for lactic acid production is imparted, as an example of lactic acid-producing bacteria, culture is usually carried out under aerobic conditions, such as shake culture or aeration agitation culture at 25° C. to 38° C. for 12 to 80 hours. During culture, the pH is preferably maintained at 2.0 to 7.0. The pH can be adjusted with an inorganic or organic acid, an alkali solution, or the like. During culture, if necessary, antibiotics such as hygromycin and G418 can be added to the medium.

Further, either a natural or synthetic medium may be used as long as it contains carbon sources, nitrogen sources, and inorganic salts that are assimilable by the microorganism, as medium compositions. Examples of carbon sources that can be used include: carbohydrates such as glucose, fructose, sucrose, starch, and cellulose; organic acids such as acetic acid and propionic acid; alcohols such as ethanol and propanol; and hydrolysates from molasses and woody biomass. Examples of nitrogen sources that can be used include: ammonia; ammonium salts comprising inorganic salts or organic acids such as ammonium chloride, ammonium sulfate, ammonium acetate, and ammonium phosphate; other nitrogen-containing compounds; peptone; meat extract; corn steep liquor; and yeast extracts. Examples of inorganic substances that can be used include monopotassium phosphate, magnesium phosphate, magnesium sulfate, sodium chloride, iron(I) sulfate, manganese sulfate, copper sulfate, and calcium carbonate. In addition, vitamins such as thiamine, biotin, folic acid, niacin, riboflavin, pyridoxine, and pantothenic acid can be added to the medium.

In addition, when using other bacteria, culture is usually carried out under conditions in which the temperature is within the range of approximately 30° C. to 60° C. for bacterial fermentation, and of approximately 20° C. to 45° C. for yeast fermentation. The temperature range for fungi fermentation is wide; however, it is within the range of approximately 20° C. to 45° C. in most cases. During culture, the pH is preferably maintained at 2.0 to 7.0. Media containing the aforementioned medium compositions can be used.

Meanwhile, when reducing the capacity for glycerol production of a lactic acid-producing microorganism according to methods 1) to 8) described above, the amount of glycerol in a solution containing lactic acid that has been generated (e.g., a medium in which lactic acid-producing bacteria are cultured) relative to the amount of lactic acid contained in the solution is preferably 3.5% by weight or less, more preferably 0.4% by weight or less, and most preferably 0.1% by weight or less.

Further, when reducing the capacity for glycerol production of a lactic acid-producing microorganism according to methods 1) to 8) described above, the amount of glycerol contained in a solution containing lactic acid that has been generated (e.g., a medium in which lactic acid-producing bacteria are cultured) relative to that of lactic acid contained in the solution must be significantly reduced compared with the amount of glycerol relative to that of lactic acid generated by a lactic acid-producing microorganism in which the capacity for glycerol production is not reduced. Preferably, reduction in such amount is 35% or more, more preferably by 90% or more, most preferably by 95% or more.

### Method 2

A method for removing glycerol produced by a lactic acid-producing microorganism comprises a step of removing glycerol produced by a fermentation method using lactic acid-producing bacteria such that the chemical reaction represented by the above chemical formula is prevented from proceeding. In addition, a solution obtained by removing cells from a culture solution of lactic acid-producing bacteria may be referred to as a crude lactic acid aqueous solution in the descriptions below.

In such step, glycerol contained in a crude lactic acid aqueous solution obtained by a fermentation method using lactic acid-producing bacteria may be removed, and glycerol contained in a culture solution of lactic acid-producing bacteria may be removed. It is desired that these steps be carried out before the chemical reaction represented by the above chemical reaction formula proceeds. Specifically, thermal energy required for the chemical reaction is added to a system in which glycerol and lactic acid coexist so that the reaction proceeds. For instance, when a crude lactic acid aqueous solution obtained by a fermentation method using lactic acid-producing bacteria is subjected to concentration by heating during a lactic acid production process, it is preferable to remove glycerol in the crude lactic acid aqueous solution prior to the concentration by heating.

After such step of removing glycerol, the amount of glycerol relative to that of lactic acid is preferably 3.5% by weight or less, more preferably 0.4% by weight or less, and most preferably 0.1% by weight or less. When the amount of glycerol relative to that of lactic acid is 3.5% by weight or less, the above chemical reaction is certainly prevented form proceeding. As a result, it is possible to achieve significantly high optical purity with respect to the lactic acid that is finally obtained. Meanwhile, when the amount of glycerol relative to that of lactic acid exceeds 3.5% by weight, the above chemical reaction proceeds. As a result, the optical purity of the lactic acid that is finally obtained is disadvantageously reduced.

More specifically, examples of a method for removing glycerol contained in a crude lactic acid aqueous solution or a culture solution include electrodialysis, an ion exchange method, chromatography, an extraction method (solvent extraction method), a centrifugation method, and a method for separating glycerol after modification into a substance that tends to be precipitated. Note that a technique for removing glycerol contained in a crude lactic acid aqueous solution or a culture solution is not limited to these methods. For instance, examples of such technique include a method wherein glycerol is subjected to chemical reaction so as to result in another substance.

Here, electrodialysis is a method wherein a pair of electrodes is disposed in a crude lactic acid aqueous solution or a culture solution, and a direct current is applied to the solution, such that lactic acid and glycerol are separated and located in the vicinities of the different electrodes, respectively. When electrodialysis is applied, for the ease of separation of lactic acid contained in a crude lactic acid aqueous solution or a culture solution, preferably, lactic acid is previously made to form lactate using alkali. An ion exchange method is a method wherein a crude lactic aqueous solution or a culture solution is applied to ion exchange resins, such that glycerol and lactic acid are separated due to use of adsorption of ionic substances on the ion exchange resins. Chromatography is a method wherein a crude lactic acid aqueous solution or a culture solution is applied together with a developer to a column such that glycerol and lactic acid can be separated as a result of differences in moving velocities of glycerol and lactic acid. An extraction method is a method wherein a solvent is used for dissolution and separation of component substances contained in a crude aqueous solution or a culture solution. A centrifugation method is a method wherein centrifugal force is applied to a crude lactic acid aqueous solution or a culture solution such that glycerol and lactic acid are separated as a result of differences in specific gravities of glycerol and lactic acid. Examples of a method for separating glycerol after modification into a substance that tends to be precipitated include: a method wherein glycerol is sulfonated by adding concentrated sulfuric acid or fuming sulfuric acid to a crude lactic acid aqueous solution or a culture solution, and sulfonated glycerol is precipitated therein, such that lactic acid and glycerol are separated by filtering the crude lactic acid aqueous solution or the culture solution; and a method wherein calcium hydroxide or calcium carbonate is added to a crude lactic acid aqueous solution or a culture solution such that lactic acid is neutralized, lactic acid is precipitated therein by cooling so as to result in calcium lactate, and then lactic acid and glycerol are separated by filtering the crude lactic acid aqueous solution or the culture solution.

Meanwhile, examples of a method wherein glycerol is subjected to chemical reaction so as to result in another substance include: a method wherein dehydration of a glycerol molecule is allowed to proceed under acidic conditions; and a method wherein glycerol and carbonyl compounds (aldehyde compounds or ketone compounds) are allowed to react with each other, resulting in the generation of acetal.

According to methods 1 and 2 described above, the amount of glycerol contained in a crude lactic acid aqueous solution or a culture solution can be reduced. The method for producing lactic acid according to the present invention comprises a step of allowing lactic acid in a solution to be subjected to concentration by heating. In such step, a solution prepared by removing cells from a culture solution obtained by method 1 or a solution in which glycerol has been removed by method 2 is subjected to concentration by heating under reduced pressure until the concentration of lactic acid contained in the solution becomes, but is not particularly limited to, approximately 60% to 70% by mass. In this method, the amount of glycerol in the solution is reduced such that the chemical reaction represented by the above chemical formula does not occur. Accordingly, lactic acid with high optical purity can be produced even after concentration by heating.

Particularly, in this method, when producing lactic acid by a fermentation method using lactic acid-producing bacteria having the capacity for L-lactic acid production, optical purity of lactic acid that is 99% or more can finally be achieved. When producing lactic acid with high optical purity even by a conventional method, it is impossible to produce lactic acid with optical purity of 99% or more, so that high optical purity desired in the present invention has not previously been achieved. Thus, preferably, lactic acid with optical purity of 99% or more serves as a starting material for polylactic acid excellent in terms of biodegradability or as a starting material for polylactic acid excellent in terms of physical properties.

In addition, according to methods 1 and 2 described above, productivity of lactic acid can be improved by reducing the amount of glycerol contained in a crude lactic acid aqueous solution or a culture solution, and lactic acid with high optical purity can be produced. For instance, in the case of a yeast (an example of lactic acid-producing bacteria) into which a lactate dehydrogenase gene is introduced, the yield of lactic acid is not necessarily high, since ethanol fermentation inherent in yeast is carried out. Thus, suppression of alcohol fermentation has been attempted for the purpose of the improvement of yield of lactic acid. However, in the case of a lactic acid-producing yeast in which alcohol fermentation is suppressed, a strain that is fully sufficient in terms of fermentation rate, cultivation rate, or the like, in addition to the yield of lactic acid, cannot be obtained.

On the other hand, according to methods 1 and 2 described above, the amount of ethanol produced can be reduced by reducing the amount of glycerol contained in a crude lactic acid solution or a culture solution. As a result, the yield of lactic acid can be improved. Thus, according to the method for producing lactic acid according to the present invention, lactic acid with high productivity and high yield that is excellent in terms of optical purity can be produced.

In addition, the method for producing lactic acid according to the present invention may comprise a processing step similar to that of a known method wherein lactic acid is produced by a fermentation method using lactic acid-producing bacteria. For instance, in such fermentation method, a lactic acid component contained in a culture solution and a crude lactic acid aqueous solution is neutralized with ammonia such that ammonium lactate is formed. Also, in the method for producing lactic acid according to the present invention, a lactic acid component contained in a culture solution and a crude lactic acid aqueous solution may be neutralized with ammonia such that ammonium lactate is formed. When ammonium lactate is contained in a culture solution and a crude lactic acid aqueous solution, after being subjected to concentration by heating described above, the lactic acid component is separated followed by esterification using alcohol such as butanol and distillation in the form of a lactate such as butyl lactate. Thereafter, the thus-separated lactate is hydrolyzed and concentrated, such that lactic acid is produced. In addition, when a lactic acid component is not neutralized with ammonia, and it is contained in a culture solution and a crude lactic acid aqueous solution in the form of lactic acid, lactic acid can be produced, followed by direct distillation from the culture solution and the crude lactic acid aqueous solution.

The present invention will hereafter be described in greater detail with reference to examples, although the technical scope of the invention is not limited thereto.

### EXPERIMENTAL EXAMPLES

Prior to describing examples to which the present invention is applied, it is verified that the reaction represented as the above formula occurs in practice. In this experimental example, it was verified that the chemical reaction between glycerol and lactic acid and the chemical reaction between ethylene glycol and lactic acid could occur in practice.

First, a solution was prepared, in which L-lactic acid was mixed with glycerol or ethylene glycol at a ratio of 1:2 (molar ratio). Then, p-toluenesulfonic acid was added to the solution, followed by heating (at 150° C. for 15 hours) under ordinary pressure while water contained in the solution was being evaporated.

After the termination of the reaction, the solution was dissolved to chloroform (1% to 10% by mass), followed by GC-MS analysis. Upon GC-MS analysis, a quadrupole mass spectrometer (JMS-AM SUN200, JEOL) and a column (DB-1, J&W Scientific) were used under the following conditions: injection temperature: 300° C.; column temperature: 50° C. to 300° C.; temperature rise rate: 5° C./min; and helium flow rate: 1 ml/min.

When the chemical reaction between glycerol and lactic acid represented by the above chemical reaction formula is in progress, a cyclic compound described in the formula can be detected. In addition, it is thought that chemical reaction represented by a chemical reaction formula described below occurs between ethylene glycol and lactic acid. Thus, a cyclic compound of the formula described below can be detected. The more the reaction that leads to generation of a cyclic compound described in the formula below progresses, the lower the optical purity of L-lactic acid.

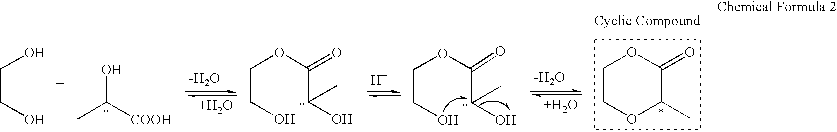

A cyclic compound resulting from a chemical reaction between glycerol and lactic acid is observed on the assumption that molecular ion peaks thereof at 146 and 115 can be simultaneously detected. This is because, in MS spectra of a glycerol dimer, which is similar to the above cyclic compound in terms of structure, molecular ion peaks are observed when a hydroxymethyl group, which is a side chain of the dimer, is removed. In addition, a cyclic compound resulting from a chemical reaction between ethylene glycol and lactic acid, which was verified as a reference example, is observed on the assumption that molecular ion peaks thereof at 116 and 73 can be simultaneously detected (Macromolecules, 2001, 34, 8641).

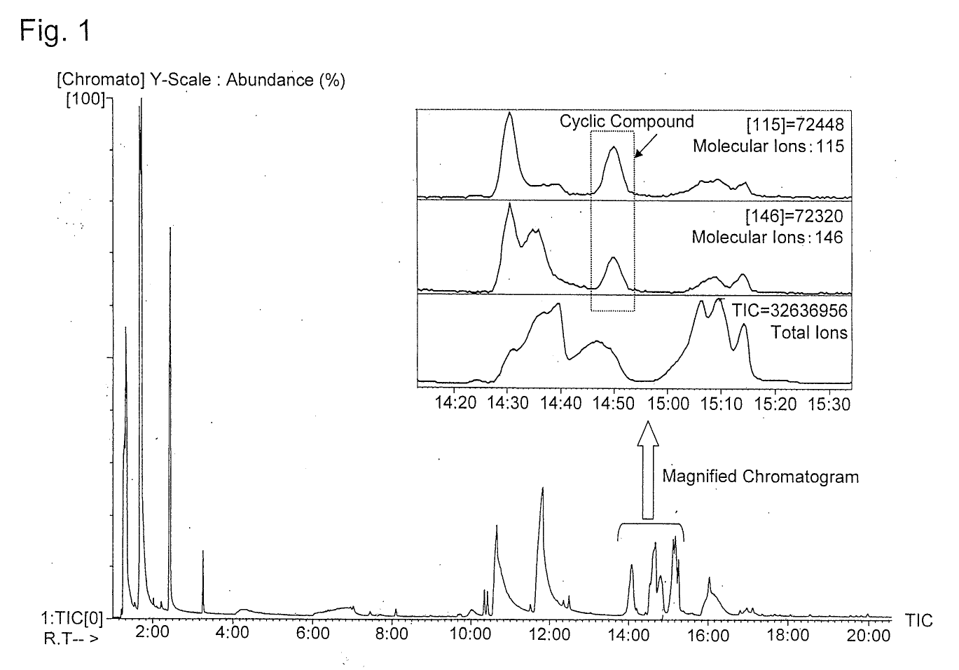

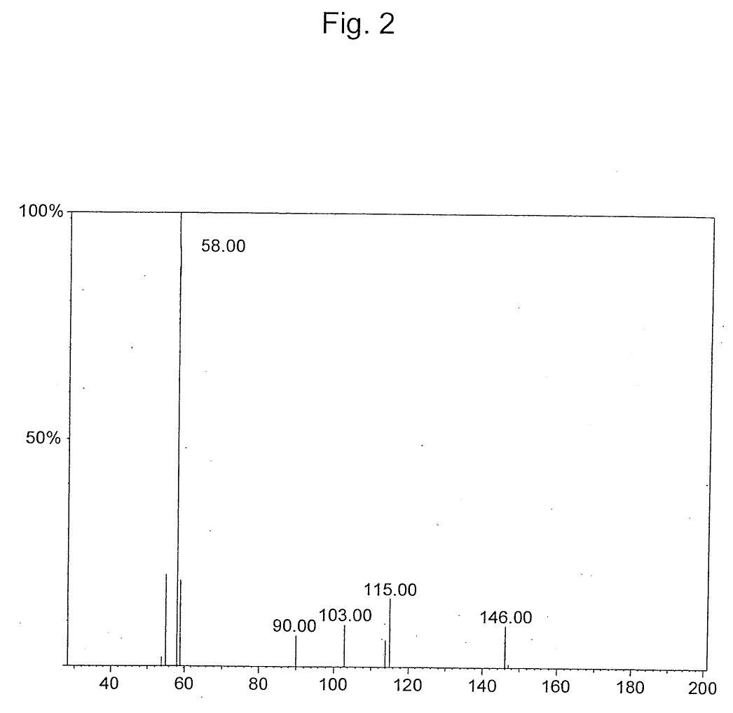

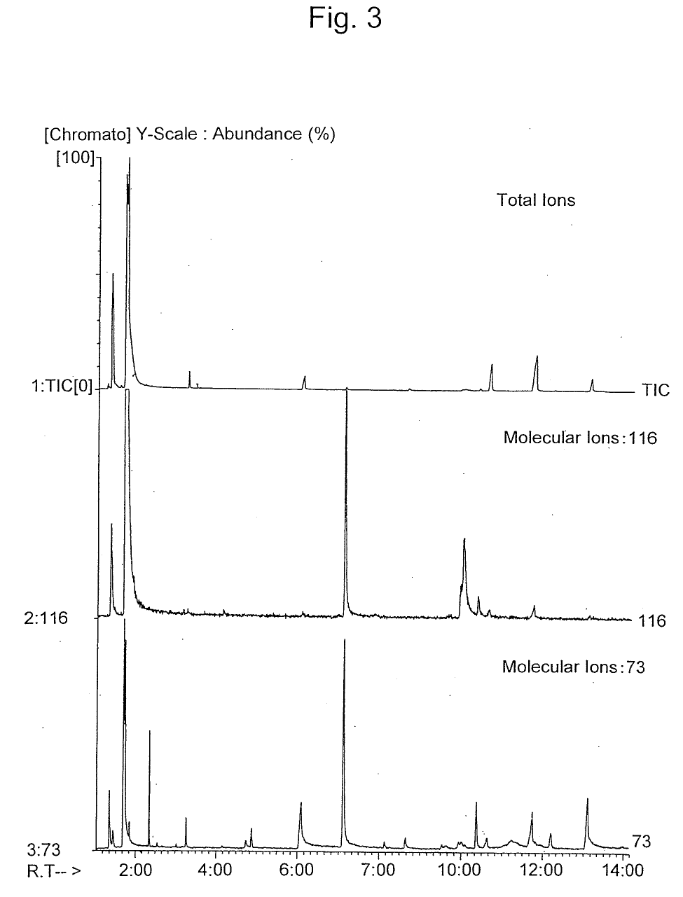

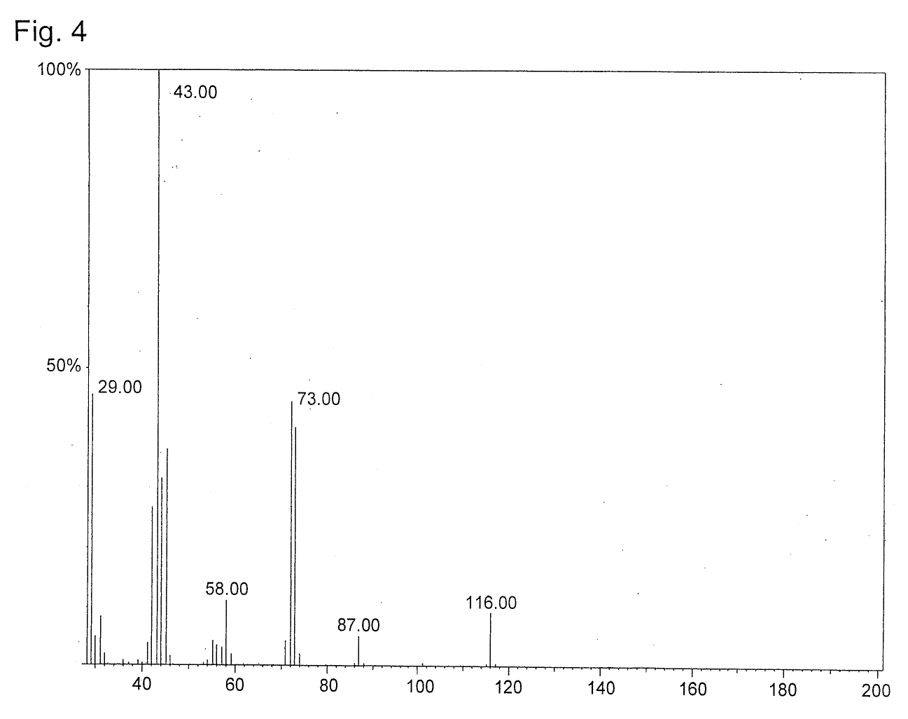

As a result of an experiment in which a solution containing L-lactic acid and glycerol was used, it was possible to observe that the resulting compound simultaneously showed molecular ion peaks at 146 and 115 at a retention time of 14.5 minutes (see FIGS. 1 and 2). In addition, as a result of an experiment in which a solution containing L-lactic acid and ethylene glycol were used, it was possible to observe that the resulting compound simultaneously showed molecular ion peaks at 73 and 116 at a retention time of 7 minutes (see FIGS. 3 and 4). Also, it was confirmed that the peak intensity ratio was almost equivalent to that described in the reference. (The intensity ratio between molecular ion peaks at 116 and 73 was 23:100 (Macromolecules, 2001, 34, 8641).)

Based on the above results, it was possible to confirm that the chemical reaction represented by the above chemical reaction formula proceeds when thermal energy is added to a system in which lactic acid and glycerol or ethylene glycol coexist. Thus, according to these experimental examples, it has been suggested that lactic acid with high optical purity can be produced by reducing the amount of glycerol in a solution prior to allowing lactic acid in the solution to be subjected to concentration by heating.

### Example 1

According to the above experimental examples, it has been suggested that lactic acid with high optical purity can be produced by reducing the amount of glycerol in a solution prior to allowing lactic acid in the solution to be subjected to concentration by heating. Thus, in this example, it was demonstrated that production of lactic acid with high optical purity was possible by a fermentation method using a lactic acid-producing microorganism, in which a gene involved in glycerol production has been disrupted.

Creation of a Strain Containing a Disrupted Gene

### Production of a Strain Containing Disrupted GPD1

A yeast having the capacity for lactic acid production that had been produced according to JP Patent Publication (Kokai) No. 2003-259878 A (JP Patent Application No. 2002-65879) was allowed to form spores in a spore-forming medium (1% potassium phosphate; 0.1% yeast extract; 0/05% dextrose; 2% agar), followed by diploidization utilizing homothallism. Then, a strain in which an LDH gene had been introduced into each diploid chromosome was obtained. The obtained strain was determined to be strain KCB-27-7.

A DNA fragment of a hygromycin resistance gene (hereafter referred to as an BPH gene) was amplified by PCR using Escherichia coli strain K12 as a template. The DNA nucleotide sequence of the HPH gene has been registered in the GenBank database with accession no. V01499. Primers used were HPH-U (5′-ATG AAA AAG CCT GAA CTC ACC-3′ (SEQ ID NO: 1)) and HPH-D (5′-CTA TTC CTT TGC CCT CGG ACG-3′ (SEQ ID NO: 2)), which were located at both ends of the HPH gene.

A DNA fragment of the TDH3 promoter region was amplified by PCR using genome DNA of yeast strain IFO2260 (registered with the Institute of Fermentation) as a template. The DNA nucleotide sequence of the TDH3 gene has been registered in the GenBank database with accession no. Z72977. Primers used were TDH3P-U (5′-ATA TAT GGA TCC TAG CGT TGA ATG TTA GCG TCA AC-3′; BamHI site-added TDH3 promoter sequence (SEQ ID NO: 3)) and TDH-3P-D (5′-ATA TAT CCC GGG TTT GTT TGT TTA TGT GTG TTT ATT CG-3′; SmaI site-added TDH3 promoter sequence (SEQ ID NO: 4)).

A DNA fragment of the CYC1 terminator region was amplified by PCR using genome DNA of yeast strain IFO2260 as a template. The DNA nucleotide sequence of the CYC1 terminator region has been registered in the GenBank database with accession no. Z49548. Primers used were CYCT-U (5′-ATA TAT AAG CTT ACA GGC CCC TTT TCC TTT G-3′; HindIII site-added CYC1 terminator sequence (SEQ ID NO: 5)) and TDH-3P-D (5′-ATA TAT GTC GAC GTT ACA TGC GTA CAC GCG-3′; SalI site-added CYC1 terminator sequence (SEQ ID NO: 5)).

A fragment of the HPH gene was inserted into the EcoRV site of Escherichia coli plasmid pBluescriptII (Promega). The resulting plasmid was designated as pBhph. The plasmid pBhph was cleaved at the BamHI and SmaI sites, and then a TDH3 promoter fragment was inserted thereinto. The resulting plasmid was designated as pBhph-P. Further, the plasmid was cleaved at the HindIII and SalI sites, and then a CYC1 terminator fragment was inserted thereinto. The resulting plasmid was designated as pBhph-PT. PCR was carried out to amplify a DNA fragment in which a part (77 bp) of the GPD1 gene was added to both ends of the HPH gene cassette, to which the TDH3 promoter region and the CYC1 terminator region had been added using pPBhph-PT as a template. The DNA nucleotide sequence of the GPD1 gene added has been registered in the GenBank database with accession no. Z24454. Primers used were GPD1-CYC1-R (5′-TTA CGT TAC CTT AAA TTC TTT CTC CCT TTA ATT TTC TTT TAT CTT ACT CTC CTA CAT AAG ACA TCA AGA AAC AAT TGg tta cat gcg tac acg cgt ttg t-3′; where uppercase letters indicate the GPD1 gene sequence and lowercase letters indicate the HPH gene sequence (SEQ ID NO: 6)), in which the region from −127 to −51 of a GPD1 gene was added to outside of the HPH gene, and GPD1-TDH3-F (5′-CTA ATC TTC ATG TAG ATC TAA TTC TTC AAT CAT GTC CGG CAG GTT CTT CAT TGG GTA GTT GTT GTA AAC GAT TTG Gta gcg ttg aat gtt agc gtc aac a-3′; where uppercase letters indicate the GPD1 gene sequence and lowercase letters indicate the HPH gene sequence (SEQ ID NO: 7)), in which the region from +1100 to +1176 of a GPD1 gene was added in a similar manner. Using the resulting PCR product, strain KCB27-7 was transformed by a lithium acetate method (Ito et al., J. Bacteriol., 153, 163-168 (1983)). After transformation, the transformed strain was inoculated into a YPD medium plate containing 200 μg/ml of hygromycin and subjected to culture at 30° C. for 2 days, resulting in the obtaining of a transformant thereof. Genome DNA was prepared from the transformant. Then, using GPD1-295F (5′-TGC TTC TCT CCC CTT CTT-3′ (SEQ ID NO: 8)) and GPD1+1472R (5′-CAG CCT CTG AAT GAG TGG T-3′ (SEQ ID NO: 9)), which were primers outside of the inserted DNA fragment, the HPH gene was confirmed by PCR to be incorporated into a chromosome in the GPD1 gene region.

The resulting strain was allowed to form spores in a spore-forming medium, followed by diploidization utilizing homothallism. Then, a strain was obtained, in which an HPH gene was incorporated into each GPD1 gene region of diploid chromosomes such that a GPD1 gene was disrupted. The obtained strain was determined to be strain TC20.

### Production of a Strain Containing Disrupted GPD2

A DNA fragment of the chloramphenicol resistance gene (hereafter to be referred to as a CAT gene) was amplified by PCR using pCAT 3-Basic Vector (Promega) as a template. The DNA nucleotide sequence of the CAT gene has been registered in the GenBank database with accession no. M16323. Primers used were CAT-U (5′-ATA TAT CCC GGG ATG GAG AAA AAA ATC ACT GGA TAT AC-3′ (SEQ ID No: 10)) and CAT-D (5′-ATA TAT AAG CTT TTA CGC CCC GCC CTG CCA CTC ATC-3′ (SEQ ID NO: 11)), which were located at both ends of the CAT gene.

A CAT gene fragment was inserted into an EcoRV site of Escherichia coli plasmid pBluescriptII (Promega). The plasmid was designated as pBCAT. The plasmid was cleaved at the BamHI and SmaI sites, and then a TDH3 promoter fragment was inserted thereinto. The resulting plasmid was designated as pBCAT-P. The plasmid was further cleaved at the HindIII and SalI sites, and then a CYC1 terminator fragment was inserted thereinto. The resulting plasmid was designated as pBCAT-PT.

PCR was carried out using pPBCAT-PT as a template to amplify a DNA fragment in which a part of a GPD2 gene was added to both ends of a CAT gene cassette to which TDH3 promoter and CYC1 terminator regions were added. The DNA nucleotide sequence of the GPD2 gene added has been registered in the GenBank database with the accession no. Z74801. Primers used were GPD2-CYC1-R (5′-ATT TAT CCT TGG GTT CTT CTT TCT ACT CCT TTA GAT TTT TTT TTT ATA TAT TAA TTT TTA AGT TTA TGT ATT TTG GTg tta cat gcg tac acg cgt ttg t-3′; where uppercase letters indicate the GPD2 gene sequence and lowercase letters indicate the CAT gene sequence (SEQ ID NO: 12)), in which the region from −127 to −51 of a GPD2 gene was added outside the CAT gene, and GPD2-TDH3-F (5′-CTA TTC GTC ATC GAT GTC TAG CTC TTC AAT CAT CTC CGG TAG GTC TTC CAT GCG GAC GTT GTT GTA GAC TAT CTG Gta gcg ttg aat gtt agc gtc aac a-3′; where uppercase letters indicate the GPD2 gene sequence and lowercase letters indicate the CAT gene sequence (SEQ ID NO: 13)), in which the region from +1247 to +1323 of a GPD2 gene was added in a similar manner. Using the resulting PCR product, strain KCB27-7 and strain TC20 were transformed by a lithium acetate method. Thereafter, the transformed strains were inoculated into a YPD medium containing 6 mg/ml of chloramphenicol, followed by cultivation at 30° C. for 2 days. Thus, the transformants were obtained. Genome DNA was prepared from the transformants. Then, the CAT gene was confirmed to be incorporated into chromosomes in the GPD2 region by PCR using primers GPD2-262F (5′-GTT CAG CAG CTC TTC TCT AC-3′ (SEQ ID NO: 14)) and GPD2+1873R (5′-CGC AGT CAT CAA TCT GAT CC-3′ (SEQ ID NO: 15)), which were outside the inserted DNA fragment.

The resulting strain was allowed to form spores in a spore-forming medium, followed by diploidization utilizing homothallism. Then, a strain was obtained, in which a CAT gene was incorporated into each GPD2 gene region of diploid chromosomes such that a GPD2 gene was disrupted. The obtained GPD2-disrupted strain was designated as strain TC21 in the case that the strain was derived from the strain KCB27-7, or as strain TC38 in the case that the strain was derived from the strain TC20.

### Fermentation Test 1

The transformants obtained above were inoculated into a 500-ml fermentation medium (sucrose: 14.4%; molasses: 0.6%) to a cell concentration of 0.3% and were subjected to fermentation at 34° C., pH 5.0 (neutralized with ammonia), and an airflow volume of 0.6 vvm for 3 days. Thereafter, the amounts of L-lactic acid and glycerol produced were examined. The concentrations of L-lactic acid, ethanol, and glycerol were determined using a biosensor BF-4 (Oji Scientific Instruments). The yield of L-lactic acid based on sugar was calculated by dividing the amount of L-lactic acid produced by the sugar content before fermentation. The results are listed in Table 1.

TABLE 1

| | L-lactic acid (%) | Glycerol (%) |
|---|---|---|
| Strain TC38 (LDH-introduced and | 9.1 | 0.0082 |
| GPD1/GPD2-disrupted strain) | | |
| Strain KCB27-7 | 8.6 | 0.64 |
| (LDH-introduced strain) | | |

As listed in Table 1, the concentrations of L-lactic acid and glycerol in the culture solution upon termination of fermentation were 9.1% by weight (equivalent to 10.8% by weight of ammonium lactate) and 0.0082% by weight, respectively. The concentration of glycerol relative to that of lactic acid was 0.1% or less. In addition, the concentration of D-lactic acid was determined using an F-kit (Roche), so that the optical purity of L-lactic acid was calculated in accordance with the following equation. In the following equation, the concentration of D-lactic acid and that of L-lactic acid are represented by “D” and “L,” respectively.

(L−D)×100/(L+D) Equation 1

As a result of the calculation, the optical purity of L-lactic acid was found to be 99.93%.

### Fermentation Test 2

Each of the transformants obtained above was inoculated in a 100-ml Erlenmeyer flask containing 50 ml of fermentation medium (glucose: 4%; yeast extract: 1%) to a cell concentration of 0.3%, and were subjected to fermentation while being shaken (revolution: 80 rmp; shaking amplitude: 70 mm) at 32° C. for 2 to 3 days. Thereafter, the amounts of L-lactic, ethanol, and glycerol produced were examined. The results are listed in Table 2.

TABLE 2

| | | | | | Amount of |
|---|---|---|---|---|---|
| | | | | Yield of | glycerol relative |
| | | | | L-lactic acid | to amount of |
| | L-lactic acid | Ethanol | Glycerol | based on sugar | L-lactic acid |
| | (%) | (%) | (%) | (%) | (%) |
| Strain TC 20 | 3.00 | 0.50 | 0.011 | 75 | 0.37 |
| (LDH-introduced and | | | | | |
| GPD1-disrupted strain) | | | | | |
| Strain TC 21 | 2.84 | 0.58 | 0.091 | 71 | 3.2 |
| (LDH-introduced and | | | | | |
| GPD2-disrupted strain) | | | | | |
| Strain TC 38 | 3.09 | 0.45 | 0.002 | 77 | 0.065 |
| (LDH-introduced and | | | | | |
| GPD1/GPD2-disrupted strain) | | | | | |
| Strain KCB27-7 | 2.65 | 0.67 | 0.14 | 66 | 5.3 |
| (LDH-introduced strain) | | | | | |

As a result, compared with the strain KCB27-7, the amount of L-lactic acid production increased and the amounts of ethanol production and glycerol production declined in the GPD1-disrupted strain and the GPD2-disrupted strain, so that the yields of L-lactic acid based on sugar were found to have improved. In the case of the GPD1 and GPD2-disrupted strain, the amounts of ethanol production and glycerol production further declined compared with those of the GPD1-disrupted strain and the GPD2-disrupted strain, so that the yield based on sugar was improved.

More specifically, compared with the amount of glycerol relative to that of lactic acid (5.3% by weight) in the strain KCB27-7, the amount of glycerol relative to that of lactic acid in the strain TC20 (0.37% by weight) decreased by 93.0%, the amount of glycerol relative to that of lactic acid in the strain TC21 (3.2% by weight) decreased by 39.6%, and the amount of glycerol relative to that of lactic acid in the strain TC38 (0.065% by weight) decreased by 98.8%.

Accordingly, it was possible to confirm that decrease in the amount of glycerol production in the GPD1-disrupted strain and/or the GPD2-disrupted strain contributes to reduction in ethanol productivity and to improvement of lactic acid productivity. In addition, it was shown that the yield of L-lactic acid based on sugar was improved by 5% or more, and by 10% or more in a preferable case.

### Purification of L-Lactic Acid

First, cells were separated from a culture solution obtained by the fermentation method described above with the use of a filter (product name: Microza; Asahi Kasei Chemicals) such that a crude lactic acid aqueous solution was prepared. Then, the obtained crude lactic acid aqueous solution was subjected to concentration by heating to 124° C. (heat source temperature: 160° C.) under atmospheric pressure, such that the concentration of L-lactic acid contained in the crude lactic acid aqueous solution was determined to be approximately 70%.

Next, butanol was added to the crude lactic acid aqueous solution, which had been subjected to concentration by heating, in an amount that was 3 times (moles) the amount of lactic acid. The resulting solution was subjected to reaction under atmospheric pressure at 110° C. to 120° C. (heat source temperature: 160° C.) for 12 hours, such that ammonium lactate contained in the crude lactic acid aqueous solution was esterified. Then, the reaction solution containing butyl lactate was distilled under conditions of a pressure of 20 torr and a temperature of 120° C. (heat source temperature: 160° C.), such that butyl lactate was separated and purified.

Thereafter, water was added to the obtained separated and purified butyl lactate in an amount that was 16 times (moles) the amount of butyl lactate. The resulting solution was subjected to reaction under atmospheric pressure at 100° C. (heat source temperature: 160° C.) for 8 hours, such that butyl lactate was hydrolyzed. Lastly, the reaction solution was subjected to concentration by heating to 128° C. (heat source temperature: 160° C.) under atmospheric pressure, such that the concentration of L-lactic acid contained in the purified lactic acid aqueous solution was determined to be approximately 90%.

L-lactic acid obtained through the above steps was determined to be a final product. The optical purity of L-lactic acid in the obtained final product was 99.51%. In addition, the recovery rate of lactic acid after the above steps was 76.0%.

### Example 2

In this example, it was demonstrated that production of lactic acid with high optical purity was possible by removing glycerol produced by lactic acid-producing bacteria, followed by allowing lactic acid to be subjected to concentration by heating.

### Lactic Acid-Producing Bacteria and Fermentation Method

A lactic acid-producing microorganism used in this example was Saccharomyces cerevisiae used in Example 1, to which the capacity for lactic acid production was imparted, except that GPD1 and GPD2 genes were not disrupted therein. Also, in this example, a fermentation method was performed under similar conditions of Example 1.

The concentrations of L-lactic acid and glycerol upon termination of fermentation were 8.6% by weight (equivalent to 10.2% by weigh of ammonium lactate) and 0.7% by weight, respectively. The optical purity of L-lactic acid was 99.71%.

### Removal of Glycerol

In this example, first, cells were separated from a culture solution obtained by the fermentation method described above with the use of a filter (product name: Microza; Asahi Kasei Chemicals) such that a crude lactic acid aqueous solution was prepared. Then, the obtained crude lactic acid aqueous solution was subjected to electrodialysis, such that glycerol in the solution was separated and removed. Specifically, an electrodialysis apparatus (MICRO ACILYZER S3: Asahi Kasei Chemicals) and a cartridge (AC-110-550: Asahi Kasei Chemicals) were used. In the apparatus, a crude lactic acid aqueous solution was placed on the dilution side and distilled water was placed on the concentration side. Electrodialysis was performed with an applied voltage of 15V until an electric conductivity of 0.5 mS was obtained on the dilution side, such that lactic acid was transferred to the concentration side. Further, the crude lactic acid aqueous solution on the dilution side, in which the electric conductivity had declined, was discarded. Then, a crude lactic acid aqueous solution was placed again on the dilution side. Electrodialysis was repeatedly performed.

As a result of determination after electrodialysis, the concentrations of L-lactic acid and glycerol contained in the crude lactic acid aqueous solution were found to be 21.6% by weight and 0.02% by weight, respectively. The concentration of glycerol relative to L-lactic acid was 0.1% or less.

### Purification of L-Lactic Acid

A crude lactic acid aqueous solution in which glycerol was removed as described above was subjected to concentration by heating to 124° C. (heat source temperature: 160° C.) under atmospheric pressure using a Rotavapor R-220 (Buchi), such that the concentration of L-lactic acid contained in the crude lactic acid aqueous solution was determined to be approximately 65%.

Next, butanol was added to the crude lactic acid aqueous solution, which had been subjected to concentration by heating, in an amount that was 3 times (moles) the amount of lactic acid. The resulting solution was subjected to reaction under atmospheric pressure at 110° C. to 120° C. (heat source temperature: 160° C.) for 12 hours, such that ammonium lactate contained in the crude lactic acid aqueous solution was esterified. Then, the reaction solution containing butyl lactate was distilled under conditions of a pressure of 20 torr and a temperature of 120° C. (heat source temperature: 160° C.), such that butyl lactate was separated and purified.

Thereafter, water was added to the obtained separated and purified butyl lactate in an amount that was 16 times (moles) the amount of butyl lactate. The resulting solution was subjected to reaction under atmospheric pressure at 100° C. (heat source temperature: 160° C.) for 8 hours, such that butyl lactate was hydrolyzed. Lastly, the reaction solution was subjected to concentration by heating to 128° C. (heat source temperature: 160° C.) under atmospheric pressure, such that the concentration of L-lactic acid contained in the purified lactic acid aqueous solution was determined to be approximately 90%.

L-lactic acid obtained through the above steps was determined to be a final product. The optical purity of L-lactic acid in the obtained final product was 99.16%. In addition, the recovery rate of lactic acid after the above steps was 64.8%.

### Comparative Example

For comparison, a fermentation method was performed as described in Example 2 using the lactic acid-producing bacteria used in Example 2. In this comparative example, glycerol contained in a crude lactic acid aqueous solution was not removed, and then the subsequent purification of L-lactic acid was performed. As a result, the optical purity of L-lactic acid contained in the culture solution after the termination of fermentation was 99.71%. The concentration of glycerol contained in the culture solution relative to that of L lactic acid was 1%. After purification of L-lactic acid, the optical purity of L-lactic acid was 98.40%. In addition, the recovery rate of L-lactic acid was 70.4%.

### [Results]

As is apparent from the results in Examples 1 and 2, it was demonstrated that production of L-lactic acid with high optical purity was possible by reducing the amount of glycerol prior to allowing L-lactic acid to be subjected to concentration by heating. More specifically, when L-lactic acid was subjected to concentration by heating in a system containing 1% glycerol (Comparative Example 1), the optical purity of L-lactic acid was 98.40%. However, when L-lactic acid was subjected to concentration by heating in a system containing 0.1% or less glycerol (Examples 1 and 2), the optical purity of L-lactic acid was 99% or more. Thus, in accordance with Examples 1 and 2, a method for producing L-lactic acid with high optical purity, such as 99% or more, was established.

All publications, patents, and patent applications cited herein are incorporated herein by reference in their entirety.

### INDUSTRIAL APPLICABILITY

According to the present invention, a method for producing lactic acid is provided, whereby it is possible to produce lactic acid with high optical purity, which is also available for use as, for example, a starting material for polylactic acid having excellent physical properties.

### Free Text of Sequence Listing

SEQ ID NOS: 1 to 15 indicate synthetic RNAs

1-15. (canceled)

16. A method for producing lactic acid, comprising a step of producing lactic acid by lactic acid fermentation using a microorganism having a reduced capacity for glycerol production.

17. The method for producing lactic acid according to claim 16, wherein the organism is a variant, in which expression of at least one gene involved in glycerol production is suppressed.

18. The method for producing lactic acid according to claim 17, wherein the variant is a lactic acid-producing microorganism, in which glycerol-3-phosphate dehydrogenase is disrupted.

19. The method for producing lactic acid according to claim 18, wherein the lactic acid-producing microorganism is a microorganism classified as a member of the genus Saccharomyces.

20. The method for producing lactic acid according to claim 18, wherein the lactic acid-producing microorganism is mutagenized such that the amount of glycerol produced relative to that of lactic acid is reduced by 3.5% by weight or more.

21. The method for producing lactic acid according to claim 20, wherein the lactic acid-producing microorganism is mutagenized such that the amount of glycerol produced relative to that of lactic acid is reduced by 0.1% by weight or more.


SEQUENCE LISTING (15 sequences)

SEQ ID NO: 1 - DNA, 21 bp, Artificial
  Synthetic DNA
atgaaaaagcctgaactcacc

SEQ ID NO: 2 - DNA, 21 bp, Artificial
  Synthetic DNA
ctattcctttgccctcggacg

SEQ ID NO: 3 - DNA, 35 bp, Artificial
  Synthetic DNA
atatatggatcctagcgttgaatgttagcgtcaac

SEQ ID NO: 4 - DNA, 38 bp, Artificial
  Synthetic DNA
atatatcccgggtttgtttgtttatgtgtgtttattcg

SEQ ID NO: 5 - DNA, 31 bp, Artificial
  Synthetic DNA
atatataagcttacaggccccttttcctttg

SEQ ID NO: 6 - DNA, 100 bp, Artificial
  Synthetic DNA
ttacgttaccttaaattctttctccctttaattttcttttatcttactctcctacataagacatcaagaaacaattggttacatgcgtacacgcgtttgt

SEQ ID NO: 7 - DNA, 100 bp, Artificial
  Synthetic DNA
ctaatcttcatgtagatctaattcttcaatcatgtccggcaggttcttcattgggtagttgttgtaaacgatttggtagcgttgaatgttagcgtcaaca

SEQ ID NO: 8 - DNA, 18 bp, Artificial
  Synthetic DNA
tgcttctctccccttctt

SEQ ID NO: 9 - DNA, 19 bp, Artificial
  Synthetic DNA
cagcctctgaatgagtggt

SEQ ID NO: 10 - DNA, 38 bp, Artificial
  Synthetic DNA
atatatcccgggatggagaaaaaaatcactggatatac

SEQ ID NO: 11 - DNA, 36 bp, Artificial
  Synthetic DNA
atatataagcttttacgccccgccctgccactcatc

SEQ ID NO: 12 - DNA, 100 bp, Artificial
  Synthetic DNA
atttatccttgggttcttctttctactcctttagattttttttttatatattaatttttaagtttatgtattttggtgttacatgcgtacacgcgtttgt

SEQ ID NO: 13 - DNA, 100 bp, Artificial
  Synthetic DNA
ctattcgtcatcgatgtctagctcttcaatcatctccggtaggtcttccatgcggacgttgttgtagactatctggtagcgttgaatgttagcgtcaaca

SEQ ID NO: 14 - DNA, 20 bp, Artificial
  Synthetic DNA
gttcagcagctcttctctac

SEQ ID NO: 15 - DNA, 20 bp, Artificial
  Synthetic DNA
cgcagtcatcaatctgatcc